(19) United States
(12) Patent Application Publication
(10) Pub. No.: US 2008/0119571 A1
(43) Pub. Date: May 22, 2008

(54) COMPOSITIONS AND METHODS TO ENHANCE REVERSE CHOLESTEROL TRANSPORT

(75) Inventors: Ish Khanna, Alpharetta, GA (US); Sivaram Pillarisetti, Norcross, GA (US); Uday Saxena, Atlanta, GA (US)

Correspondence Address: Reddy Us Therapeutics, Inc, 3065 Northwoods Circle, Norcross, GA, 30071, US

(73) Assignee: REDDY US THERAPEUTICS, INC., NORCROSS, GA (US)

(21) Appl. No.: 11/759,713

(22) Filed: Jun. 7, 2007

Related U.S. Application Data
(60) Provisional application No. 60/811,669, filed on Jun. 7, 2006.

(51) Int. Cl. A61K 45/00; A61P 3/00; A61P 9/00; C12N 5/00

(52) U.S. Cl. 514/789; 435/11; 435/375

(57) ABSTRACT

The present invention shows that pharmacological up regulation of SRB-1, ABC-A1 and LCAT genes collectively can be used to promote reverse cholesterol transport and increase circulating HDL cholesterol as treatment for atherosclerosis and related cardiovascular disorders.

### CROSS-REFERENCE TO RELATED PATENTS AND PATENT APPLICATIONS

The present application is a U.S. Non-provisional of and claims the priority benefit of U.S. Provisional Application No. 60/811,669, filed 7 Jun. 2006, which is relied on herein and incorporated herein by reference in its entirety.

### BACKGROUND OF THE INVENTION

Cardiovascular disease continues to be the leading cause of mortality in industrialized nations. See Gotto A M Jr., J Am Coll Cardiol. 2005; 46(7):1219-24. Atherosclerosis or clogging of arteries is the major cause of cardiovascular events such as myocardial ischemia, stroke and death. See Libby P., et al., Circulation. 111(25):3481-8 (2005). Several clinical trials using statins for both primary and secondary prevention have shown a marked reduction in coronary events, mainly owing to the lowering of plasma concentrations of low-density lipoprotein (LDL) cholesterol. See Gotto A M Jr., et al., Clin Cardiol. 2005; 28(11):499-503 and Ballantyne C M, Tex Heart Inst J. 2005; 32(3):378-9. However, recent studies also show the limitation of statin monotherapy in inhibiting the development of established atherosclerotic disease, and there is therefore there is urgent need to devise other approaches for the treatment of atherosclerosis. Especially there is now great focus on the role of other lipid factor such as high-density lipoproteins (HDL). See Olsson A G, et al., Eur Heart J. 2005; 26(9):890-6, Nicholls S J, et al., Eur Heart J. 2005; 26(9):853-5 and Duffy D, et al., Circulation. 2006; 113(8):1140-50.

The inverse relationship between plasma levels of HDL and CHD has been demonstrated in a number of epidemiological studies. See Duffy D, et al., Circulation. 2006; 113(8):1140-50, Krasuski R A, Curr Opin Lipidol. 2005; 16(6):652-7, Linsel-Nitschke P, et al., Nat Rev Drug Discov. 2005; 4(3):193-205 and Ashen M D, et al., N Engl J Med. 2005; 353(12):1252-60. In Framingham Heart Study involving 2815 men and women between the ages 49 and 82, HDL level was found to be a potential risk factor, especially among the subjects of older ages. The inverse relationship between HDL and the risk of CHD was further supported by Prospective Cardiovascular Munster (PROCAM) and the Quebec Cardiovascular Studies, which suggest that HDL cholesterol can be used as a predictor for CAD.

The importance of HDL was also highlighted from the results of intervention trials [8-10]. See Linsel-Nitschke P, et al., Nat Rev Drug Discov. 2005; 4(3):193-205 and Ashen M D, et al., N Engl J Med. 2005; 353(12):1252-60. In the High-Density Lipoprotein Intervention Trial (VA-HIT), a total of 2531 men with coronary heart disease, with mean HDL cholesterol 0.82 mmol/L (31.5 mg/dL) were randomized to gemfibrozil or placebo and were followed up for 5 years. The reduction in risk (stroke) was evident after 6 to 12 months and patients with baseline HDL cholesterol below the median appear to benefit more from treatment than those with higher HDL cholesterol. The effect of HDL levels on cardiovascular risks was also examined in the Helsinki Heart Study, in which 4081 asymptomatic middle-aged men with primary dyslipidemia were treated with gemfibrozil, and major event rates were monitored in a period of 5 years. It is evident from this study that the increased HDL by gemfibrozil is associated with a 34% decrease in CAD.

HDL cholesterol is beneficial largely because of its ability to perform reverse cholesterol transport, i.e. scavenge excess cholesterol from the artery and deposit it in the liver for clearance through bilary excretion. See Zannis V I, et al., J Mol Med. 2006; 84(4):276-94, Lewis G F, et al., Circ Res. 2005; 96(12):1221-32 and Tall A R, et al., Arterioscler Thromb Vasc Biol. 2000; 20(5):1185-8. HDL also exhibits other ant-inflammatory and antioxidant properties beneficial in treatment of atherosclerosis. See Barter P J, et al., Circ Res. 2004; 95(8):764-72. HDL is the smallest (7.0-12 nm diameter) and densest of the plasma lipoproteins. They consist of a hydrophobic core composed mainly of cholesteryl esters plus a small amount of triglyceride (TG) and unesterified cholesterol surrounded by a surface monolayer of phospholipids, unesterified cholesterol and apolipoproteins. The main HDL apolipoproteins (apo) are apoA-I and apoA-II. Other apolipoproteins also associate with HDL and they include apoA-IV, apoA-V, apoC-I, apoC-II, apoCIII, apoD, apoE, apoJ, and apoL and enzymes involved in lipid metabolism, including, cholesteryl ester transfer protein (CETP), lecithin:cholesterol acyltransferase (LCAT) and phospholipid transfer protein (PLTP) (11, 12). See Zannis V I, et al., J Mol Med. 2006; 84(4):276-94 and Lewis G F, et al., Circ Res. 2005; 96(12):1221-32. The atheroprotective effects of HDL to a large extent are attributed to its ability to remove cholesterol from macrophages present in atherosclerotic lesions to transport them to liver for excretion into bile, a process termed Reverse Cholesterol Transport (RCT). [11, 12]. See Zannis V I, et al., J Mol Med. 2006; 84(4):276-94 and Lewis G F, et al., Circ Res. 2005; 96(12):1221-32. ApoA1 is mainly synthesized in the liver (Step 1). Cholesterol/phospholipids are transferred to free ApoA1 and lipid-poor ApoA1 forming prep or discoid HDL. This process is facilitated by transport protein ABCA1 present in liver (Step 2a) as well as macrophages present in atherosclerotic lesions (Step 2b). ABCA1 can transfer phospholipids to nascent ApoA1 or cholesterol esters to A1-phospholipid particles (discoidal HDL). The cholesterol in HDL particles is esterified by LCAT (Step 3) to cholesterol esters resulting in a particle with a core of cholesteryl esters (spherical HDL). Most of the HDL particles in plasma are spherical. The classic model of RCT involves esterification of cholesterol by LCAT and uptake by the liver in the esterified form (step 5b). HDL cholesterol is transported to the liver through interaction with the scavenger receptor, class B, type I (SR-BI). SR-BI selectively takes up cholesterol from HDL particles leaving cholesterol-depleted A1 particles in circulation. HDL cholesterol that is taken up by the liver is then excreted in the form of bile acids and cholesterol (Step 6), completing the process of reverse cholesterol transport. From the above description, it is clear that SRB-1, ABC-A1 and LCAT are essential and rate-limiting proteins in the reverse cholesterol pathway. See Zannis V I, et al., J Mol Med. 2006; 84(4):276-94. Simultaneous up regulation of these genes/proteins should provide the greathe test enhancement of reverse cholesterol transport pathways.

There are currently no drugs on the market that directly increase the reverse cholesterol pathway. See Ashen M D, et al., N Engl J Med. 2005; 353(12):1252-60. Pathways targeted by industry to increase HDL have been to increase synthesis and secretion of apoAI, the major protein in HDL and/or decrease HDL catabolism. Several known agents such as Gemfibrozil increase HDLc levels. See Linsel-Nitschke P, et al., Nat Rev Drug Discov. 2005; 4(3): 193-205. Gemfibrozil is a member of an important class of drugs called fibrates that act on the liver. Fibrates are fibric acid derivatives (bezafibrate, fenofibrate, gemfibrozil and clofibrate) which profoundly lower plasma triglyceride levels and elevate HDLc. The typical clinical use of fibrates is in patients with hypertriglyceridemia, low HDLc and combined hyperlipidemia. The mechanism of action of fibrates involves the induction of certain apolipoproteins and enzymes involved in VLDL and HDL metabolism. See Staels B, et al., Diabetes 2005, 54:2460-2470 and Meyers C D, et al., Curr Opin Cardiol. 2005; 20(4):307-12.

Nicotinic acid (niacin), a water-soluble vitamin has a lipid lowering profile similar to fibrates and may target the liver. Niacin has been reported to increase apoAI by selectively decreasing hepatic removal of HDL apoAI, but niacin does not increase the selective hepatic uptake of cholesteryl esters. See Meyers C D, et al., Curr Opin Cardiol. 2005; 20(4):307-12. In addition, premenopausal women have significant cardio-protection as a result of high HDLc levels, probably due to estrogens. See Rossouw J E, Curr Opin Lipidol. 1999; 10(5):429-34.

Dexamethasone, prednisone, and estrogen activate the apoAI gene, increase apoAI and HDL cholesterol, reduce lipoprotein B, and reduce LDL. The side effects of such steroids are well known and limit their chronic use in atherosclerosis.

Currently, there are no pharmacological agents that coordinately up-regulate all three critical genes involved in RCT, namely, the ABCA1, SR-B1 and LCAT genes.

Therefore, an object of the invention is to provide compounds, compositions and methods that lead to increase in reverse cholesterol transport and in circulating HDL levels. It would also be desirable, therefore, to develop a screening method to identify compounds that upregulate the activity of ACBA1, LCAT, and SRB1, and therefore, the rate of reverse cholesterol transport. Compounds that are identified by this method would be useful for raising HDL and for prevention and/or treatment of diseases associated with cholesterol deposition and transport.

### SUMMARY OF INVENTION

Because reverse cholesterol transport can be a powerful therapeutic approach to atherosclerosis disease modification and since there are no drugs that promote these pathways directly, the current invention provides methods, compounds and composition to up regulate SRB-1, ABC-A1 and LCAT genes in a concerted fashion. Simultaneous up-regulation of all three genes will for maximal enhancement of reverse cholesterol transport as opposed to modulating just one of these genes/proteins. The up regulation of these genes can be used as a monotherapy or could also be used in combination therapy with other lipid regulating agents such as statins, fibrates or niacin. In addition, the RCT gene up regulation approach can also be used with the direct disease modifying approaches currently in clinical trials.

These and other aspects of the invention will be understood and become apparent upon review of the specification by those having ordinary skill in the art.

### BRIEF DESCRIPTION OF THE FIGURES

FIG. 1 shows a diagram depicting the critical role of ABCA1, SR-B1 and LCAT in reverse cholesterol transport (RCT) pathway.

FIG. 2 shows that compound A induces ABCA1 and SRB1 expression in LDL-r null mouse liver.

FIG. 3 shows that compound A induces LCAT expression in LDL-r null mouse liver.

FIG. 4 shows that compound A induces ABCA1 expression in rat liver.

FIG. 5 shows that compound A induces SRB1 expression in rat liver.

FIG. 6 shows that compound A induces ABCA1 expression in rat liver cell line.

FIG. 7 shows that compound A induces SRB1 expression in rat liver.

FIG. 8 shows that compound A inhibits atherosclerosis development in LDL-r null mice.

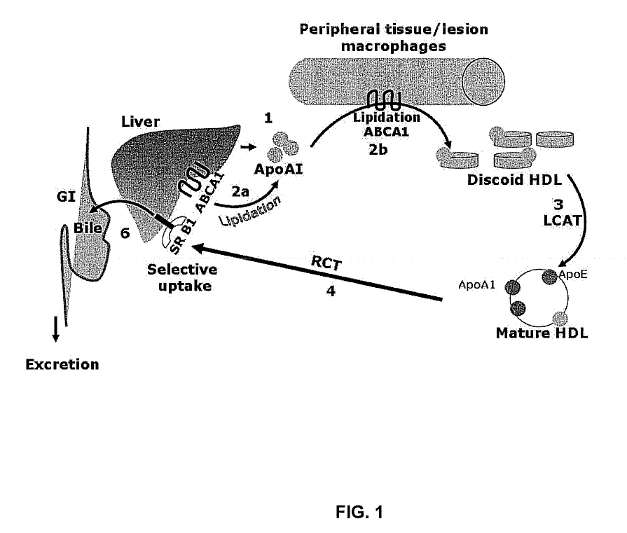

### DETAILED DESCRIPTION OF THE PREFERRED EMBODIMENTS

Reference now will be made in detail to the embodiments of the invention, one or more examples of which are set forth below. Each example is provided by way of explanation of the invention, not limitation of the invention. In fact, it will be apparent to those skilled in the art that various modifications and variations can be made in the present invention without departing from the scope or spirit of the invention. For instance, features illustrated or described as part of one embodiment can be used on another embodiment to yield a still further embodiment. Thus, it is intended that the present invention covers such modifications and variations as come within the scope of the appended claims and their equivalents. Other objects, features and aspects of the present invention are disclosed in or are obvious from the following detailed description. It is to be understood by one of ordinary skill in the art that the present discussion is a description of exemplary embodiments only, and is not intended as limiting the broader aspects of the present invention.

It has been shown that, surprisingly, an upregulation in the activity of the RCT pathway genes, namely, ABCA1, LCAT, and SRB1 results in an increase in the activity of the RCT pathway. As is known to those having ordinary skill in the art, an increase in the activity of the RCT pathway serves to raise HDL levels, and preventing and/or reducing plaque build-up in arteries. In one aspect, therefore, the invention is a method of increasing the activity of the ABCA1, LCAT, and SRB1 genes, and thereby increasing the RCT pathway, raising levels of HDL and preventing and/or reducing plaque build-up in arteries.

To determine if a test compound was capable of upregulating these three RCT pathway genes, LDL-r mice were treated with vehicle alone (control) or vehicle containing a defined amount of a test compound termed, “compound A”.

As used herein, “compound A” has the structure:

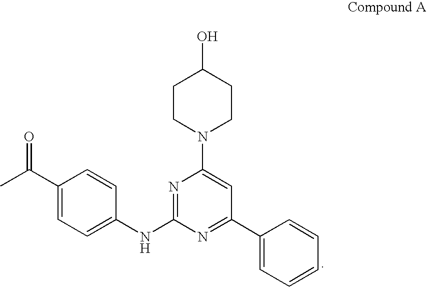

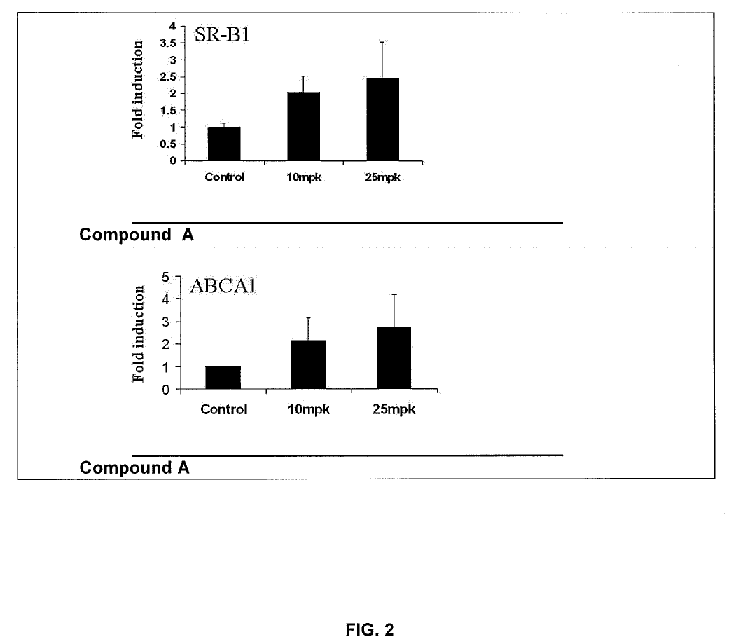

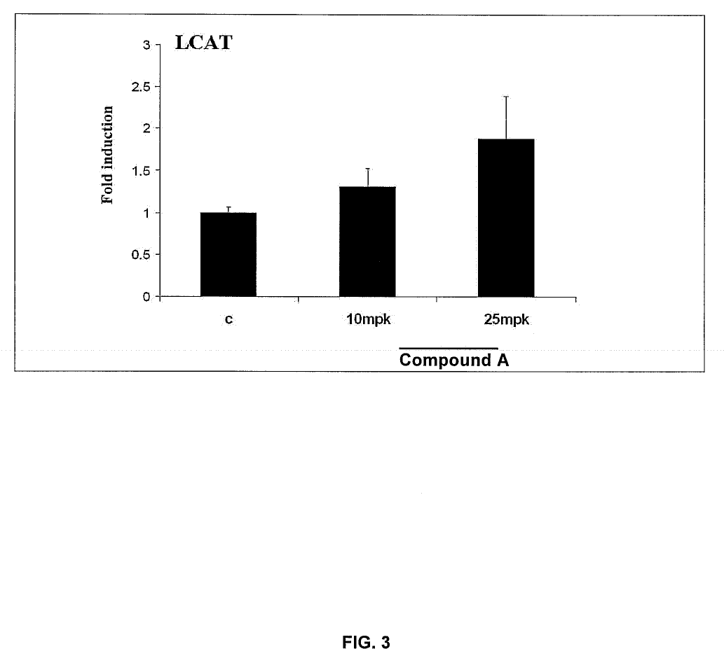

LDL-r mice were chosen because they are prone to develop atherosclerosis. After 7 days of treatment, the three RCT pathway genes were quantitated using real time PCR. As shown in FIG. 2 compared to vehicle treated animals, compound A at 10 and 25 mg/kg resulted in a 2-2.5 fold increase in SR-B1 and 2-3 fold increase in ABCA1 expression. In addition, compound A also increased LCAT expression by 30-90% (FIG. 3). Thus, compound A coordinately up regulated the three RCT pathway genes, namely ABCA1, LCAT, and SRB1.

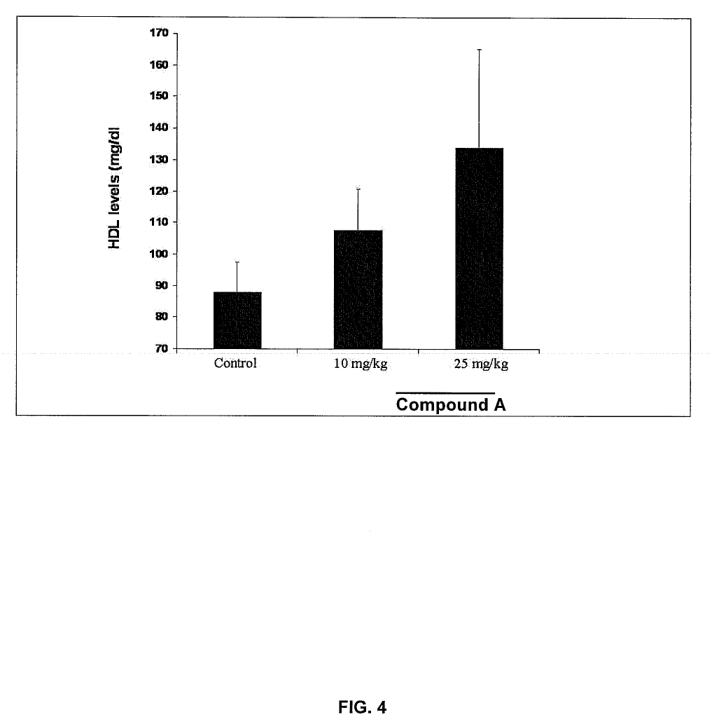

Upregulation of ABCA1 and LCAT also should result in the formation of HDL particles. Following 7-day treatment, compound A increased HDL levels by greater than 50% (FIG. 4).

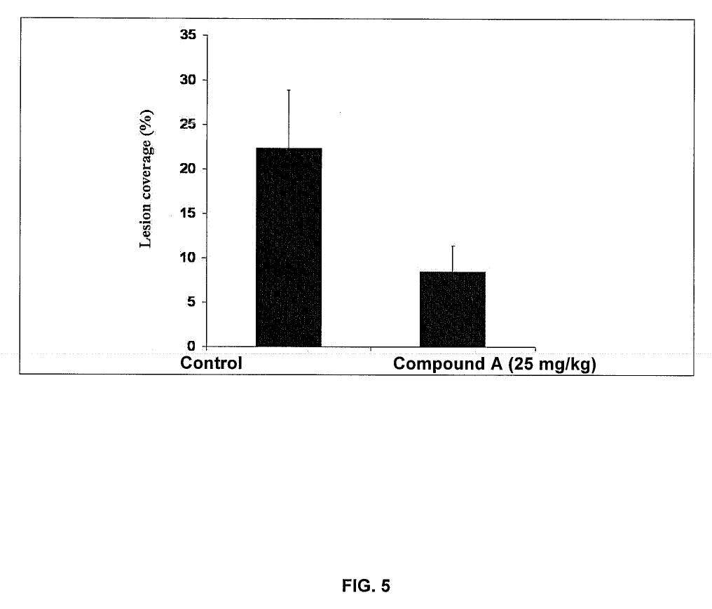

The atheroprotective effects of HDL to a large extent are attributed to its ability to remove cholesterol from macrophages present in atherosclerotic lesions to transport them to liver for excretion into bile. LDLr null mice develop atherosclerosis when fed a high fat diet. Treatment with compound A led to significant decrease in atherosclerotic lesions (FIG. 5).

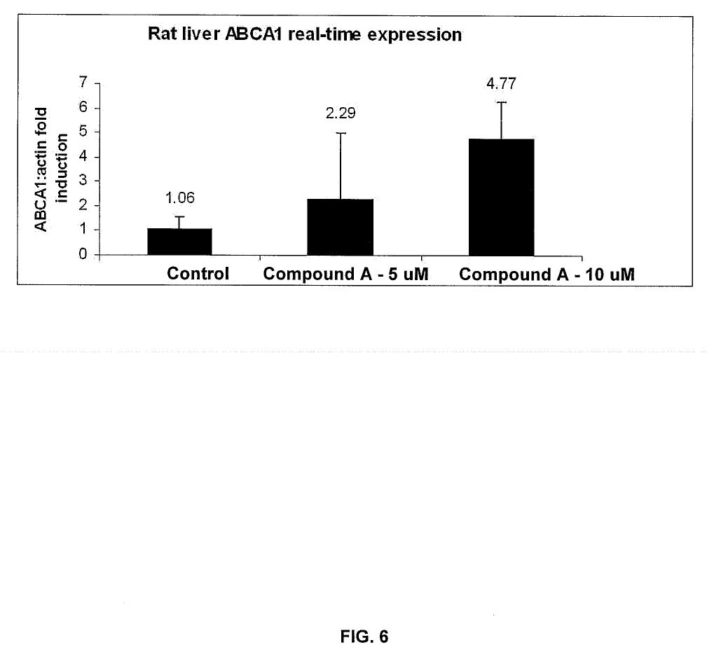

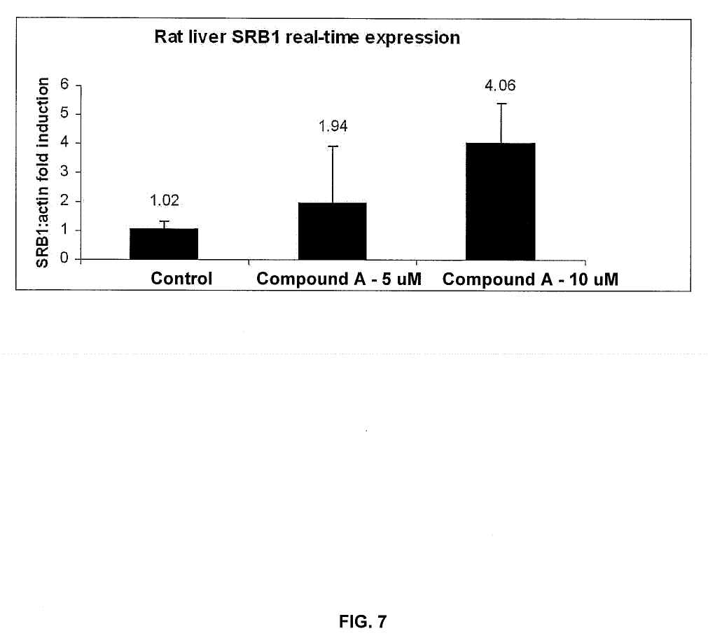

Compound A also increased ABCA1 and SR-B1 expression in normal animals. After 7 days of treatment RCT pathway genes were quantitated using real time PCR. As shown in FIGS. 6 and 7 compared to vehicle treated animals, compound A at 10 and 25 mg/kg resulted in a 2-4 fold increase in SR-B1 and ABCA1 expression.

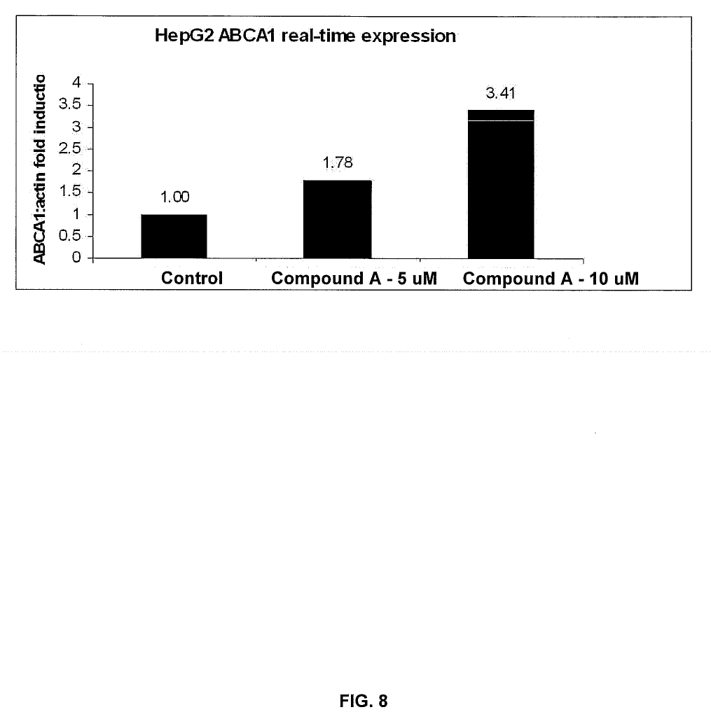

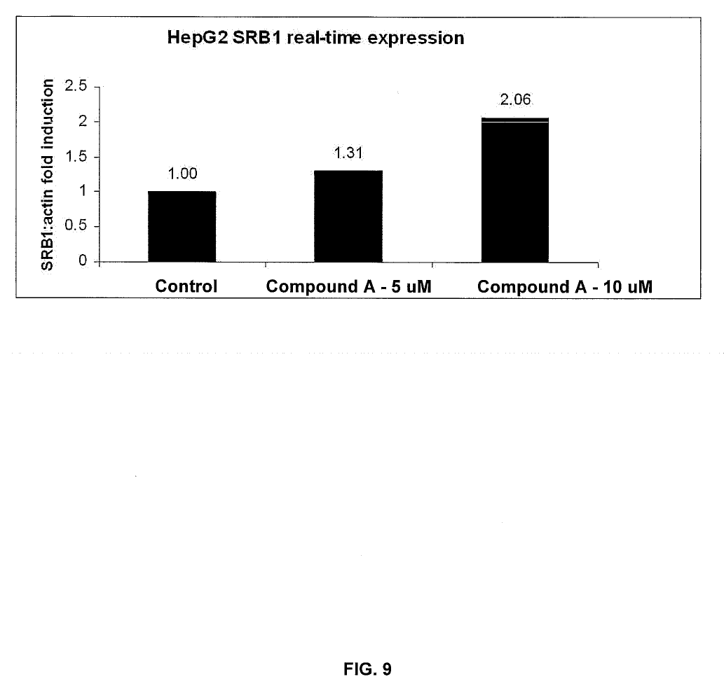

The hepatoma cell line HepG2 are frequently used as in vitro models of liver cells. They are easy to culture and retain many properties of liver cells including expression of various genes involved in lipid metabolism. At least one compound, namely, the compound of Formula I, is capable of increasing ABCA1 and SRB1 expression in liver cells (FIGS. 8, 9).

Therefore, in one aspect, the invention is a method of identifying compounds that are capable of up regulating the activity of the ABCA1, SR-B1 and LCAT genes. The method includes providing a sample of cells that express the three genes, providing a sample of an ABCA1, SR-B1 and LCAT gene activity-modulating test compound (a “test compound”), contacting the cell sample and the test compound sample in the presence of an assay for ABCA1, SR-B1 and LCAT activity, and measuring the change in ABCA1, SR-B1 and LCAT activity that is caused by the contact with the test compound. The upregulation or change in activity of the ABCA1, SR-B1 and LCAT genes can occur at the transcriptional level or at the translational level or both.

It has been found to be useful to use a high-throughput assay based on real-time PCR for the assay for ABCA1, SR-B1 and LCAT activity as the measure of a quantitative indicator of the change in activity within the cell sample that is caused by the test compound. It has also been found to be useful to use a high-throughput assay based on luciferase activity for the assay for ABCA1, SR-B1 and LCAT activity as the measure of a quantitative indicator of the change in activity within the cell sample that is caused by the test compound. In one embodiment, the cells that express ABCA1, SR-B1 and LCAT are human liver cells.

In a preferred embodiment, the present invention is a method of identifying compounds capable of upregulating ABCA1, SR-B1 and LCAT activity.

In preferred embodiments, the quantitative indicator of ABCA1, SR-B1 and LCAT is luciferase-construct activity or real-time transcript PCR activity. In one embodiment, the step of contacting the cell sample and the test sample in the presence of a high-throughput assay based on luciferase activity includes contacting the cell sample and the test sample in which the luciferase gene is joined to a ABCA1, SR-B1 or LCAT promoter in an expression vector that is transfected into cells. When the sample candidate successfully upregulates the ABCA1, SR-B1 or LCAT activity, expression of the luciferase reporter is increased and measured through an enzymatic release of light. In these embodiments, the quantitative activity that is measured is the light given off by the expressed luciferase.

The quantitative indicator may also be one or more post-translational activities, including, but not limited to phosphorylation, localization, or acetylation.

In the present screening method, an increase in the monitored quantitative indicator indicates an upregulation of ABCA1, SR-B1 or LCAT activity. As used herein, the terms “ABCA1, SR-B1 or LCAT activity” refer to the amount of or concentration of an ABCA1, SR-B1 or LCAT RNA transcript and/or the activity of an ABCA1, SR-B1 or LCAT polypeptide in the modulation of the RCT pathway. Accordingly, in the present method, when the monitored quantitative indicator indicates an increase in ABCA1, SR-B1 or LCAT activity upon contact of the cell sample with the test compound, the test compound is shown to be effective in upregulating the ABCA1, SR-B1 or LCAT activity.

In another aspect, the invention is a method of increasing HDL levels by administering to a subject an RCT inducer. As used herein, the term “RCT inducer” will be understood by those having ordinary skill in the art as including any compound that increases the activity of the ABCA1, SR-B1 and LCAT genes. By way of example, any compound that causes an increase in ABCA1, SR-B1 and LCAT activity in the present method of identifying RCT inducers that is described herein, is considered to be an ABCA1, SR-B1 and LCAT inducer.

In certain embodiments, the present invention encompasses a method of promoting reverse cholesterol transport (RCT) in a subject, the method comprising administration of an effective amount of an agent that up regulates reverse cholesterol transporter genes. In certain embodiments, the up regulated RCT genes are SR-B1 and ABC-A1. In other embodiments, the up regulated RCT genes are SR-B1, ABC-A1, and LCAT. In certain embodiments, the agent is a synthetic organic molecule, and in some embodiments, has a molecular weight of <700. In certain embodiments, the subject is a SR-B1, ABC-A1 expressing mammal, and in particular, can be a human.

In certain embodiments, the present invention encompasses a method of increasing HDL in a subject, the method comprising administration of an effective amount of an agent that up regulates RCT genes.

In certain embodiments, the present invention encompasses a method of treating dyslipidemia in a subject, the method comprising administration of an effective amount of an agent that up regulates RCT genes.

In certain embodiments, the present invention encompasses a method of treating cardiovascular disease in a subject, the method comprising administration of an effective amount of an agent that up regulates RCT genes.

In certain embodiments, the present invention encompasses a method of up regulating SR-B1 and ABC-A1 in a cell, the method comprising contacting the cell with an effective amount of an agent that up regulates RCT genes.

In certain embodiments, the present invention encompasses a method of identifying compounds that increases HDL in a subject by up regulating RCT genes.

In certain embodiments, the present invention encompasses a method of identifying compounds that treat atherosclerosis by up regulating RCT genes.

In certain embodiments, the present invention encompasses a method of identifying compounds that treat cardiovascular disorders by up regulating RCT genes.

In certain embodiments, the present invention encompasses a method of treating atherosclerosis in a subject, the method comprising administrating an effective amount of a compound that up regulates RCT genes, wherein the administration is in combination with a CETP-inhibitor.

In certain embodiments, the present invention encompasses a method of increasing HDL in a subject, the method comprising administrating an effective amount of compound that up regulates RCT genes, wherein the administration is in combination with a CETP-inhibitor.

For ease of reference, the present invention will be described with reference to administration to human subjects. It will be understood, however, that such descriptions are not limited to administration to humans, but will also include administration to other animals, such as mammals, unless explicitly stated otherwise.

The present method includes administering one or more RCT inducers to the subject by administration means known in the art. Administration means contemplated as useful include one or more of topically, buccally, intranasally, orally, intravenously, intramuscularly, sublingually, and subcutaneously. Other administration means known in the art are also contemplated as useful in accordance with the present invention and are discussed in more detail below.

In some embodiments, it may be useful to include one or more of the RCT inducers as a salt. Those having ordinary skill in the art will recognize the salts of the RCT inducer compounds.

In some embodiments, the composition may be an aqueous composition. The composition may also be nebulized or aerosolized.

The subject invention involves the use of a safe and effective amount of one or more RCT inducers for activating the Reverse Cholesterol Transport pathway, thereby treating or preventing atherosclerosis and other conditions caused by low levels of HD in subjects having low levels of HDL, subjects having plaque-build-up in arteries, subjects suffering from atherosclerosis, and subjects in need of prevention of atherosclerosis.

One method of administering one or more RCT inducers is topical, intranasal administration, e.g., with nose drops, nasal spray, or nasal mist inhalation. Other exemplary methods of administration include one or more of topical, bronchial administration by inhalation of vapor and/or mist or powder, orally, intravenously, intramuscularly, and subcutaneously.

Other ingredients which may be incorporated in the present invention include safe and effective amounts of preservatives, e.g., benzalkonium chloride, thimerosal, phenylmercuric acetate; and acidulants, e.g., acetic acid, citric acid, lactic acid, and tartaric acid. The present invention may also include safe and effective amounts of isotonicity agents, e.g., salts, such as sodium chloride, and more preferably non-electrolyte isotonicity agents such as sorbitol, mannitol, and lower molecular weight polyethylene glycol.

In the present method, a subject in need of activating RCT is treated with an amount of one or more RCT inducers, where the amount of the one or more RCT inducers provides a dosage or amount that is sufficient to constitute a treatment or prevention effective amount.

As used herein, an “effective amount” means the dose or amount of a RCT inducer to be administered to a subject and the frequency of administration to the subject which is readily determined by one of ordinary skill in the art, by the use of known techniques and by observing results obtained under analogous circumstances and has some therapeutic action. The dose or effective amount to be administered to a subject and the frequency of administration to the subject can be readily determined by one of ordinary skill in the art by the use of known techniques and by observing results obtained under analogous circumstances. In determining the effective amount or dose, a number of factors are considered by the attending diagnostician, including but not limited to, the potency and duration of action of the compounds used; the nature and severity of the illness to be treated as well as on the sex, age, weight, general health and individual responsiveness of the subject to be treated, and other relevant circumstances.

The phrase “therapeutically-effective” indicates the capability of an agent to prevent, or improve the severity of, the disorder, while avoiding adverse side effects typically associated with alternative therapies.

The one or more RCT inducers can be supplied in the form of a novel therapeutic composition that is believed to be within the scope of the present invention.

When the one or more RCT inducers are supplied along with a pharmaceutically acceptable carrier, a pharmaceutical composition is formed. A pharmaceutical composition of the present invention is directed to a composition suitable for the prevention or treatment of the disorders described herein. The pharmaceutical composition comprises at least a pharmaceutically acceptable carrier and one or more RCT inducers. Pharmaceutically acceptable carriers include, but are not limited to, physiological saline, Ringer's, phosphate solution or buffer, buffered saline, and other carriers known in the art. Pharmaceutical compositions may also include stabilizers, anti-oxidants, colorants, and diluents. Pharmaceutically acceptable carriers and additives are chosen such that side effects from the pharmaceutical compound are minimized and the performance of the compound is not canceled or inhibited to such an extent that treatment is ineffective.

The term “pharmacologically effective amount” shall mean that amount of a drug or pharmaceutical agent that will elicit the biological or medical response of a tissue, system, animal or human that is being sought by a researcher or clinician. This amount can be a therapeutically effective amount.

The term “pharmaceutically acceptable” is used herein to mean that the modified noun is appropriate for use in a pharmaceutical product. Pharmaceutically acceptable cations include metallic ions and organic ions. More preferred metallic ions include, but are not limited to, appropriate alkali metal salts, alkaline earth metal salts and other physiological acceptable metal ions. Exemplary ions include aluminum, calcium, lithium, magnesium, potassium, sodium and zinc in their usual valences. Preferred organic ions include protonated tertiary amines and quaternary ammonium cations, including in part, trimethylamine, diethylamine, N,N′-dibenzylethylenediamine, chloroprocaine, choline, diethanolamine, ethylenediamine, meglumine (N-methylglucamine) and procaine. Exemplary pharmaceutically acceptable acids include, without limitation, hydrochloric acid, hydroiodic acid, hydrobromic acid, phosphoric acid, sulfuric acid, methanesulfonic acid, acetic acid, formic acid, tartaric acid, maleic acid, malic acid, citric acid, isocitric acid, succinic acid, lactic acid, gluconic acid, glucuronic acid, pyruvic acid oxalacetic acid, fumaric acid, propionic acid, aspartic acid, glutamic acid, benzoic acid, and the like.

Also included in present invention are the isomeric forms and tautomers and the pharmaceutically-acceptable salts of RCT inducers. Illustrative pharmaceutically acceptable salts are prepared from formic, acetic, propionic, succinic, glycolic, gluconic, lactic, malic, tartaric, citric, ascorbic, glucuronic, maleic, fumaric, pyruvic, aspartic, glutamic, benzoic, anthranilic, mesylic, stearic, salicylic, p-hydroxybenzoic, phenylacetic, mandelic, embonic (pamoic), methanesulfonic, ethanesulfonic, benzenesulfonic, pantothenic, toluenesulfonic, 2-hydroxyethanesulfonic, sulfanilic, cyclohexylaminosulfonic, algenic, β-hydroxybutyric, galactaric and galacturonic acids.

Suitable pharmaceutically-acceptable base addition salts of compounds of the present invention include metallic ion salts and organic ion salts. More preferred metallic ion salts include, but are not limited to, appropriate alkali metal (Group IA) salts, alkaline earth metal (Group IIA) salts and other physiological acceptable metal ions. Such salts can be made from the ions of aluminum, calcium, lithium, magnesium, potassium, sodium and zinc. Preferred organic salts can be made from tertiary amines and quaternary ammonium salts, including in part, trimethylamine, diethylamine, N,N′-dibenzylethylenediamine, chloroprocaine, choline, diethanolamine, ethylenediamine, meglumine (N-methylglucamine) and procaine. All of the above salts can be prepared by those skilled in the art by conventional means from the corresponding compound of the present invention.

The terms “treating” or “to treat” means to alleviate symptoms, eliminate the causation either on a temporary or permanent basis, or to prevent or slow the appearance of symptoms. The term “treatment” includes alleviation, elimination of causation of or prevention of any of the diseases or disorders described above. Besides being useful for human treatment, these combinations are also useful for treatment of mammals, including horses, dogs, cats, rats, mice, sheep, pigs, etc.

The term “subject” for purposes of this application includes any animal. The animal is typically a human. A preferred subject is one in need of treatment or prevention of the disorders discussed herein.

For methods of prevention, the subject is any human or animal subject, and preferably is a subject that is in need of prevention and/or treatment of atherosclerosis or other disorders caused by low levels of HDL. The subject may be a human subject who is at risk of disorders such as those described above. The subject may be at risk due to genetic predisposition, sedentary lifestyle, diet, exposure to disorder-causing agents, exposure to pathogenic agents and the like.

The present pharmaceutical compositions may be administered enterally and/or parenterally. Parenteral administration includes subcutaneous, intramuscular, intradermal, intramammary, intravenous, and other administrative methods known in the art. Enteral administration includes solution, tablets, sustained release capsules, enteric coated capsules, syrups, beverages, foods, and other nutritional supplements. When administered, the present pharmaceutical composition may be at or near body temperature.

The phrase “therapeutically-effective” and “effective for the treatment, prevention, or inhibition,” are intended to qualify the amount of each agent for use in the therapy which will achieve the goal of increased proteoglycan levels, while avoiding adverse side effects typically associated with alternative therapies.

In particular, the RCT inducers of the present invention, or compositions in which they are included, can be administered orally, for example, as tablets, coated tablets, dragees, troches, lozenges, aqueous or oily suspensions, dispersible powders or granules, emulsions, hard or soft capsules, or syrups or elixirs. Compositions intended for oral use may be prepared according to any method known in the art for the manufacture of pharmaceutical compositions and such compositions may contain one or more agents selected from the group consisting of sweetening agents, flavoring agents, coloring agents and preserving agents in order to provide pharmaceutically elegant and palatable preparations. Tablets contain the active ingredient in admixture with non-toxic pharmaceutically acceptable excipients which are suitable for the manufacture of tablets. These excipients may be, for example, inert diluents, such as calcium carbonate, sodium carbonate, lactose, calcium phosphate or sodium phosphate; granulating and disintegrating agents, for example, maize starch, or alginic acid; binding agents, for example starch, gelatin or acacia, and lubricating agents, for example magnesium stearate, stearic acid or talc. The tablets may be uncoated or they may be coated by known techniques to delay disintegration and adsorption in the gastrointestinal tract and thereby provide a sustained action over a longer period. For example, a time delay material such as glyceryl monostearate or glyceryl distearate may be employed.

Formulations for oral use may also be presented as hard gelatin capsules wherein the active ingredients are mixed with an inert solid diluent, for example, calcium carbonate, calcium phosphate or kaolin, or as soft gelatin capsules wherein the active ingredients are present as such, or mixed with water or an oil medium, for example, peanut oil, liquid paraffin, any of a variety of herbal extracts, milk, or olive oil.

Aqueous suspensions can be produced that contain the active materials in admixture with excipients suitable for the manufacture of aqueous suspensions. Such excipients are suspending agents, for example, sodium carboxymethylcellulose, methylcellulose, hydroxypropylmethyl-cellulose, sodium alginate, polyvinylpyrrolidone gum tragacanth and gum acacia; dispersing or wetting agents may be naturally-occurring phosphatides, for example lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyoxyethylene sorbitan monooleate.

The aqueous suspensions may also contain one or more preservatives, for example, ethyl or n-propyl p-hydroxybenzoate, one or more coloring agents, one or more flavoring agents, or one or more sweetening agents, such as sucrose or saccharin.

Oily suspensions may be formulated by suspending the active ingredients in an omega-3 fatty acid, a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol.

Sweetening agents, such as those set forth above, and flavoring agents may be added to provide a palatable oral preparation. These compositions may be preserved by the addition of an antioxidant such as ascorbic acid.

Dispersible powders and granules suitable for preparation of an aqueous suspension by the addition of water provide the active ingredient in admixture with a dispersing or wetting agent, a suspending agent and one or more preservatives. Suitable dispersing or wetting agents and suspending agents are exemplified by those already mentioned above. Additional excipients, for example sweetening, flavoring and coloring agents, may also be present.

Syrups and elixirs containing one or more RCT inducers may be formulated with sweetening agents, for example glycerol, sorbitol or sucrose. Such formulations may also contain a demulcent, a preservative, and flavoring and coloring agents.

The subject RCT inducers and compositions in which they are included can also be administered parenterally, either subcutaneously, or intravenously, or intramuscularly, or intrasternally, or by infusion techniques, in the form of sterile injectable aqueous or olagenous suspensions. Such suspensions may be formulated according to the known art using those suitable dispersing or wetting agents and suspending agents which have been mentioned above, or other acceptable agents. The sterile injectable preparation may also be a sterile injectable solution or suspension in a non-toxic parenterally-acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or diglycerides. In addition, n-3 polyunsaturated fatty acids may find use in the preparation of injectables.

The subject RCT inducers and compositions in which they are included can also be administered by inhalation, in the form of aerosols or solutions for nebulizers, or rectally, in the form of suppositories prepared by mixing the drug with a suitable non-irritating excipient which is solid at ordinary temperature but liquid at the rectal temperature and will therefore melt in the rectum to release the drug. Such materials are cocoa butter and poly-ethylene glycols.

The subject RCT inducers and compositions in which they are included can also be administered topically, in the form of creams, ointments, jellies, collyriums, solutions, patches, or suspensions.

Daily dosages of the RCT inducers can vary within wide limits and will be adjusted to the individual requirements in each particular case. In general, for administration to adults, an appropriate daily dosage has been described above, although the limits that were identified as being preferred may be exceeded if expedient. The daily dosage can be administered as a single dosage or in divided dosages.

Various delivery systems in addition to nutritional supplements include sprays, capsules, tablets, drops, and gelatin capsules, for example.

Those skilled in the art will appreciate that dosages for the therapeutic use of the RCT inducers may also be determined with guidance from Goodman & Goldman's The Pharmacological Basis of Therapeutics, Ninth Edition (1996), Appendix II, pp. 1707-1711.

Preferred dosages for the RCT inducers are those that are effective to increase the rate of RCT. In especially preferred embodiments, the dosage should be in a concentration effective to increase the rate of RCT such that plaque build-up in the arteries is reduced. In yet another embodiment an effective dosage is an amount that is effective to increase HDL levels in the subject. In another embodiment, an effective dosage is an amount that is effective to upregulate RCT gene activity (i.e., the ABCA1, SR-B1 and LCAT genes) in the subject.

### Example 1

This example illustrates the effect of compound A on HDL and the ABCA1, SR-B1 and LCAT genes associated with RCT in LDL-receptor (LDLr) null mice in a short term model.

Eight-weeks-old LDLr mice (Jackson Laboratories, Bar Harbor, Me.), were used for this Example. Mice were distributed into two groups of 8 mice each. One group received vehicle containing carboxymethyl cellulose and Tween-80 (Sigma Chemical Co.), and the other group received a daily dose of compound A at 25 mg/kg.

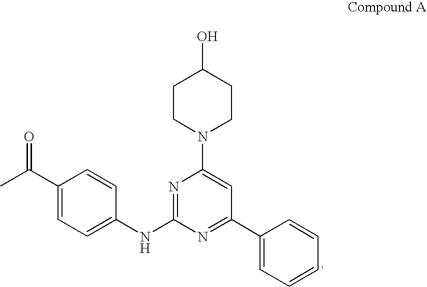

After one week, plasma samples were collected and used for HDL determination. Mice were euthanized by CO₂, and aorta and liver samples were collected for RNA isolation.

Next, liver and aorta samples from vehicle and compound A treated mice were removed, flash frozen in liquid nitrogen and subsequently used for RNA isolation. Tissues were lysed in 600 uL lysis buffer (Qiagen) and placed in the TissueLyser (Qiagen) for 3 minutes. Samples were then processed using the RNeasy mini kit (liver) or the RNeasy fibrous tissue mini kit (aorta). RNA was then verified and quantified using the Agilent RNA 600 Nano Assay Labchip® system, and real time PCR was performed to quantitate the gene expression of SR-B1, ABCA1 and LCAT using validated primer sets from SuperArray. The results were illustrated in FIGS. 2-3.

### Example 2

This example illustrates the effect of compound A on genes associated with RCT in HepG2 cells.

Human HepG2 cells were cultured in DMEM containing 4500 mg glucose/L (ATCC) supplemented with 10% fetal bovine serum (FBS), penicillin (100 U/ml), and streptomycin (100 μg/ml) in a humidified atmosphere (5% CO2 in air) at 37° C. Cells were plated onto 12 well dishes and cultured in low serum (0.5% FBS) for experiments. Cells were treated with either vehicle (DMSO) or the test compound overnight and used for RNA isolation. Briefly, cells were lysed in 300 uL of lysis buffer (Qiagen) and placed in the TissueLyser (Qiagen) for 3 minutes. Samples were then processed as described by the RNeasy mini kit. RNA was then verified and quantified using the Agilent RNA 600 Nano Assay Labchip® system, and real time PCR was performed to quantitate gene expression using validated primer sets from SuperArray. The results were illustrated in FIGS. 8-9.

### Example 3

This example illustrates the effect of compound A on HDL and atherosclerosis in LDLr null mice in a long term model.

Eight-weeks-old LDLr null mice (Jackson Laboratories, Bar Harbor, Me.), were used for these studies. Two groups of 8 mice each received a Western Diet (Research Diet incorporated, New Brunswick, N.J.; D12079B containing 0.1% cholesterol) for 2 weeks. Plasma samples were collected and used for lipid analysis. Mice were then distributed into two groups, one group received vehicle containing carboxymethyl cellulose and Tween-80 (Sigma Chemical Co.), and the other group received a daily dose of compound A at 25 mg/kg. Both groups were continued on the Western Diet for another 16 weeks. At the end of the 16 weeks, the mice were euthanized by CO₂, and whole aortas (from aortic sinus to the beginning of iliac aorta) were isolated, and fixed in 10% paraformaldehyde. The aortas were then opened and stained with Sudan IV solution for 30 minutes. Images of whole aortas were obtained by microscopic digital camera, and the lesion area was determined by computer-assisted morphometry (Image Pro, Media cybermetics) as percentages of coverage in whole aorta. The results were illustrated in FIG. 5.

### Example 4

This example illustrates the effect of compound A on lipid and atherosclerosis in an ApoE null mice model.

Eight-weeks-old ApoE null mice (Jackson Laboratories, Bar Harbor, Me.), were used for these studies. Two groups of 8 mice each received a normal chow Diet (Research Diet incorporated, New Brunswick, N.J.; D12079B containing 0.1% cholesterol) for 8 weeks. Plasma samples were collected and used for lipid analysis. Mice were then distributed into two groups, one group received vehicle containing carboxymethyl cellulose and Tween-80 (Sigma Chemical Co.), and the other group received a daily dose of the TEST compound at 25 mg/kg. Both groups continued on the normal chow diet for another 8 weeks. At the end of 8 weeks, the mice were euthanized by CO₂, and their whole aortas (from aortic sinus to the beginning of iliac aorta) were isolated, processed, and lesion areas were determined as described above.

In view of the above, it will be seen that the several advantages of the invention are achieved and other advantageous results obtained.

All references cited in this specification, including without limitation all papers, publications, patents, patent applications, presentations, texts, reports, manuscripts, brochures, books, internet postings, journal articles, periodicals, and the like, are hereby incorporated by reference into this specification in their entireties. The discussion of the references herein is intended merely to summarize the assertions made by their authors and no admission is made that any reference constitutes prior art. Applicants reserve the right to challenge the accuracy and pertinency of the cited references.

As various changes could be made in the above methods and compositions by those of ordinary skill in the art without departing from the scope of the invention, it is intended that all matter contained in the above description and shown in the accompanying drawings shall be interpreted as illustrative and not in a limiting sense. In addition it should be understood that aspects of the various embodiments may be interchanged both in whole or in part.

What is claimed:

1. A method of promoting reverse cholesterol transport in a subject, the method comprising administration of an effective amount of an agent that up regulates reverse cholesterol transporter genes.

2. A method of claim 1, wherein the up regulated reverse cholesterol transporter genes are SR-B1 and ABC-A1.

3. A method of claim 1, wherein the up regulated reverse cholesterol transporter genes are SR-B1, ABC-A1, and LCAT.

4. A method of claim 1 when the agent is a synthetic organic molecule.

5. A method of claim 1 when the synthetic organic molecule has a molecular weight of <700.

6. A method of claim 1 wherein the subject is a SR-B1, ABC-A1 expressing mammal.

7. A method of claim 6, wherein mammal is a human.

8. A method of increasing HDL in a subject, the method comprising administration of an effective amount of an agent that up regulates reverse cholesterol transporter genes.

9. A method of claim 8, wherein the up regulated reverse cholesterol transporter genes are SR-B1 and ABC-A1.

10. A method of claim 8, wherein the up regulated reverse cholesterol transporter genes are SR-B1, ABC-A1, and LCAT.

11. A method of claim 8 when the agent is a synthetic organic molecule.

12. A method of claim 8 when the synthetic organic molecule has a molecular weight of <700.

13. A method of claim 8 wherein subject is a SR-B1, ABC-A1 expressing mammal.

14. A method of claim 13, wherein mammal is a human.

15. A method of treating dyslipidemia in a subject, the method comprising administration of an effective amount of an agent that up regulates reverse cholesterol transporter genes.

16. A method of claim 15, wherein the up regulated reverse cholesterol transporter genes are SR-B1 and ABC-A1 genes.

17. A method of claim 15, wherein the up regulated reverse cholesterol transporter genes are SR-B1, ABC-A1, and LCAT genes.

18. A method of claim 15 when the agent is a synthetic organic molecule.

19. A method of claim 15 when the synthetic organic molecule has a molecular weight of <700.

20. A method of claim 15 wherein subject is a SR-B1, ABC-A1 expressing mammal.

21. A method of claim 20, wherein mammal is a human.

22. A method of treating cardiovascular disease in a subject, the method comprising administration of an effective amount of an agent that up regulates reverse cholesterol transporter genes.

23. A method of claim 22, wherein the up regulated reverse cholesterol transporter genes are SR-B1 and ABC-A1.

24. A method of claim 23, wherein the up regulated reverse cholesterol transporter genes are SR-B1, ABC-A1, and LCAT.

25. A method of claim 23 when the agent is a synthetic organic molecule.

26. A method of claim 23 when the synthetic organic molecule has a molecular weight of <700.

27. A method of claim 23 wherein subject is a SR-B1, ABC-A1 expressing mammal.

28. A method of claim 27, wherein mammal is a human.

29. A method of claim 22, wherein cardiovascular disease is atherosclerosis.

30. A method of up regulating SR-B1 and ABC-A1 in a cell, the method comprising contacting the cell with an effective amount of an agent that up regulates reverse cholesterol transporter genes.

31. A method of claim 30, wherein the cell is a macrophage or a liver cell.

32. A method of identifying compounds that up regulate reverse cholesterol transporter genes in a cell.

33. A method of claim 32 wherein the cell is a macrophage or a liver cell.

34. A method of claim 32 wherein the compound is a synthetic organic molecule with molecular weight of <700.

35. A method of claim 32, wherein the up regulated reverse cholesterol transporter genes are SR-B1 and ABC-A1.

36. A method of claim 32, wherein the up regulated reverse cholesterol transporter genes are SR-B1, ABC-A1, and LCAT.

37. A method of identifying compounds that increase HDL in a subject by up regulating reverse cholesterol transporter genes.

38. A method of claim 37 wherein compound is a synthetic organic molecule with molecular weight of <700.

39. A method of claim 37 wherein the up regulated reverse cholesterol transporter genes are SR-B1 and ABC-A1.

40. A method of claim 37, wherein the up regulated reverse cholesterol transporter genes are SR-B1, ABC-A1, and LCAT.

41. A method of identifying compounds that treat atherosclerosis by up regulating reverse cholesterol transporter genes.

42. A method of claim 41 wherein compound is a synthetic organic molecule with molecular weight of <700.

43. A method of claim 41, wherein the up regulated reverse cholesterol transporter genes are SR-B1 and ABC-A1.

44. A method of claim 41, wherein the up regulated reverse cholesterol transporter genes are SR-B1, ABC-A1, and LCAT genes.

45. A method of identifying compounds that treat cardiovascular disorders by up regulating reverse cholesterol transporter genes.

46. A method of claim 45, wherein compound is a synthetic organic molecule with molecular weight of <700.

47. A method of claim 45, wherein the up regulated reverse cholesterol transporter genes are SR-B1 and ABC-A1.

48. A method of claim 45, wherein the up regulated reverse cholesterol transporter genes are SR-B1, ABC-A1, and LCAT.

49. A method of treating atherosclerosis in a subject, the method comprising administrating an effective amount of compound of claim 41 with a CETP-inhibitor.

50. A method of increasing HDL in a subject, the method comprising administrating an effective amount of compound of claim 37 with a CETP-inhibitor.
